(19) United States
(12) Patent Application Publication
(10) Pub. No.: US 2008/0077044 A1
(43) Pub. Date: Mar. 27, 2008

(54) THERMAL SENSATION ANALYZING DEVICE, METHOD, AIR-CONDITIONING CONTROL DEVICE, METHOD, AND COMPUTER PROGRAM PRODUCT

(75) Inventors: Kanako NAKAYAMA, Kanagawa (JP); Takuji SUZUKI, Kanagawa (JP); Kazushige OUCHI, Kanagawa (JP); Kenichi KAMEYAMA, Kanagawa (JP)

Correspondence Address: OBLON, SPIVAK, MCCLELLAND MAIER & NEUSTADT, P.C., 1940 DUKE STREET, ALEXANDRIA, VA, 22314, US

(73) Assignee: Kabushiki Kaisha Toshiba, Tokyo (JP)

(21) Appl. No.: 11/687,753

(22) Filed: Mar. 19, 2007

(30) Foreign Application Priority Data
Sep. 21, 2006 (JP) 2006-256271

(51) Int. Cl. A61B 5/00; F25B 29/00

(52) U.S. Cl. 600/549; 165/253

(57) ABSTRACT

A thermal sensation analyzing device includes a first detecting unit, a second detecting unit, and an analyzing unit. The first detecting unit detects skin temperature of a person. The second detecting unit detects a predetermined cyclic-fluctuation pattern in the skin temperature. The analyzing unit analyzes thermal sensation of the person based on the cyclic-fluctuation pattern.

### CROSS-REFERENCE TO RELATED APPLICATIONS

This application is based upon and claims the benefit of priority from the prior Japanese Patent Application No. 2006-256271, filed on Sept. 21, 2006; the entire contents of which are incorporated herein by reference.

### BACKGROUND OF THE INVENTION

1. Field of the Invention

The present invention relates to a technology for analyzing thermal sensation of a person, and controlling air condition.

2. Description of the Related Art Research and development have been proceeding on a thermal sensation analyzing device that analyzes thermal sensation of every individual based on biosignals from the individual. The thermal sensation analyzing device attracts attention as a device that can easily analyze the thermal sensation compared to using thermal indices such as Standard Effective Temperature (SET*) and Predicted Mean Vote (PMV). The thermal indices such as SET* and PMV require many types of measuring equipment with respect to environmental factors such as temperature, humidity, air flow, radiant heat, amount of clothing, and work load.

In analysis of thermal sensation using biosignals, generally, skin temperature is used as an index. For example, a technology for analyzing thermal sensation based on experimental values of average skin temperature of seven different parts of a body, deep body temperature, and skin surface heat flow. Reference may be had to, for example, a paper written by A. Ishiguro et al., entitled “Indoor climate for comfortable sleep, considering heat and moisture transfer between a bedroom, bedding, and a human body: Air control system using a predictive model for thermal comfort”, “The third International Conference on Human-Environment system (ICHES'5)”, Tokyo, September 2005, pp. 139-144. Another reference may be found in a paper written by I. Mori et al., entitled “Experimental study related to thermal sensation prediction in an unsteady state”, Journal of Architecture, Planning and Environmental Engineering (Transactions of Architectural Institute of Japan) January 2003, volume 563, pp 9-15.

Another known technology is related to thermal sensation prediction based on skin temperature alone. For example, JP-A H06-265189 (KOKAI) discloses a technology for analyzing thermal sensation by measuring skin temperature at 10 different parts of a human body every 10 minutes. JP-A H11-190545 (KOKAI) discloses a technology for analyzing thermal sensation from fuzzy input of a difference between average skin temperature during one minute and that calculated one minute before.

However, in the conventional technologies disclosed in the papers “Indoor climate for comfortable sleep, considering heat and moisture transfer between a bedroom, bedding, and a human body: Air control system using a predictive model for thermal comfort” and “Experimental study related to thermal sensation prediction in an unsteady state”, many sensors need to be attached to a person's body for measuring average skin temperature and skin surface heat flow. Further, thermometers need to be inserted into a person's body to measure deep body temperature. Both these factors make it difficult to conduct thermal sensation analysis in daily activities.

Moreover, in the conventional technology disclosed in JP-A H06-265189 (KOKAI) requires quite a large area to measure temperature of ten different parts, which poses difficulty in the measurement in daily life. Infrared sensors can be an alternative for unrestrained measurement. However, the area of activity is restricted by the placement of the infrared sensors. In the conventional technology disclosed in JP-A H11-190545 (KOKAI), the fuzzy input value is derived from reading during short duration. Therefore, there is a possibility of a wrong result due to disturbance such as sudden changes in temperature.

### SUMMARY OF THE INVENTION

According to an aspect of the present invention, a thermal sensation analyzing device includes a first detecting unit that detects skin temperature of a person, a second detecting unit that detects a predetermined cyclic-fluctuation pattern in the skin temperature, and an analyzing unit that analyzes thermal sensation of the person based on the cyclic-fluctuation pattern.

According to another aspect of the present invention, a thermal sensation analyzing method includes acquiring skin temperature of a person, detecting a predetermined cyclic-fluctuation pattern in the skin temperature, and analyzing thermal sensation of the person based on the cyclic-fluctuation pattern.

According to still another aspect of the present invention, an air-conditioning control device includes a first detecting unit that detects skin temperature of a person, a second detecting unit that detects a predetermined cyclic-fluctuation pattern in the skin temperature, an analyzing unit that analyzes thermal sensation of the person based on the cyclic-fluctuation pattern, and an air-conditioning control unit that controls air-conditioning based on an analysis result obtained by the analyzing unit.

According to still another aspect of the present invention, an air-conditioning control method includes acquiring skin temperature of a person, detecting a predetermined cyclic-fluctuation pattern in the skin temperature, analyzing thermal sensation of the person based on the cyclic-fluctuation pattern, and controlling air-conditioning based on an analysis result obtained at the analyzing.

According to still another aspect of the present invention, a computer program product includes a computer usable medium having computer readable program codes embodied in the medium that, when executed, cause a computer to implement the above method.

### BRIEF DESCRIPTION OF THE DRAWINGS

FIG. 1 is a block diagram of a thermal sensation analyzing system according to a first embodiment of the present invention;

FIG. 2 is an exterior view of the thermal sensation analyzing system;

FIG. 3A is a graph of a result of skin temperature measurement;

FIG. 3B is a graph of values of thermal sensation felt by a person obtained with the measurement result shown in FIG. 3A;

FIG. 4 is a graph for explaining ripple detecting process performed by a thermal sensation analyzing unit shown in FIG. 1;

FIG. 5 is a flowchart of a thermal sensation analyzing process performed by a thermal sensation analyzing device shown in FIG. 1;

FIG. 6 is a block diagram of a hardware configuration of the thermal sensation analyzing device;

FIG. 7 is a block diagram of a thermal sensation analyzing system according to a second embodiment of the present invention;

FIG. 8 is a schematic for explaining a process performed by a gradient detecting unit shown in FIG. 7;

FIG. 9 is a flowchart of a thermal sensation analyzing process performed by a thermal sensation analyzing device shown in FIG. 7;

FIG. 10 is a flowchart of a thermal sensation analyzing process performed by a thermal sensation analyzing device according to a third embodiment of the present invention;

FIG. 11 is a block diagram of an air-conditioning control system according to a fourth embodiment of the present invention;

FIG. 12 is a flowchart of an air-conditioning process performed by an air-conditioning control device shown in FIG. 11;

FIG. 13 is a block diagram of an air-conditioning control system according to a fifth embodiment of the present invention;

FIG. 14 a flowchart of an air-conditioning process performed by an air-conditioning control device shown in FIG. 13; and

FIG. 15 is a block diagram of an air-conditioning control system according to a sixth embodiment of the present invention.

### DETAILED DESCRIPTION OF THE INVENTION

Exemplary embodiments of the present invention are explained in detail below with reference to the accompanying drawings.

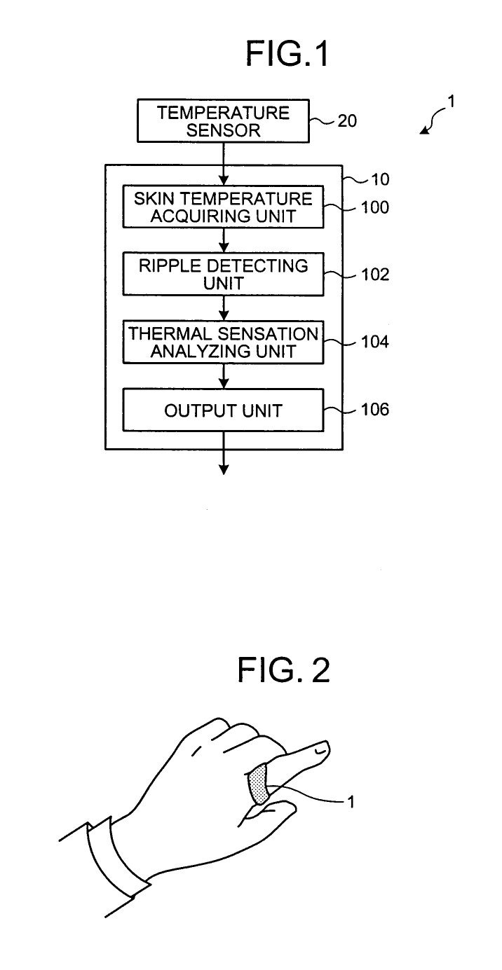

As shown in FIG. 1, according to a first embodiment of the present invention, a thermal sensation analyzing system 1 includes a thermal sensation analyzing device 10, and a temperature sensor 20. The thermal sensation analyzing device 10 includes a skin temperature acquiring unit 100, a ripple detecting unit 102, a thermal sensation analyzing unit 104, and an output unit 106.

The skin temperature acquiring unit 100 continuously acquires skin temperature from the temperature sensor 20. The ripple detecting unit 102 detects a cyclic variation, a ripple, in the skin temperature acquired by the skin temperature acquiring unit 100. The term “ripple” as used herein refers to variation in skin temperature in the form of ripples, and the cycle is of relatively short duration. Specifically, a ripple with a cycle of not less than 30 seconds and not more than 180 seconds is detected by frequency analysis based on Fourier transform. The thermal sensation analyzing unit 104 analyzes person's thermal sensation based on ripples detected by the ripple detecting unit 102 and the skin temperature. The output unit 106 outputs a result of the thermal sensation analysis by the thermal sensation analyzing unit 104. Processes performed by the thermal sensation analyzing unit 104 and the output unit 106 are described later.

As shown in FIG. 2, the thermal sensation analyzing system 1 is attached to a person on, for example, his/her finger, to detect his/her skin temperature. The thermal sensation analyzing system 1 shown in FIG. 2 is in the form of a ring. The temperature sensor 20 is arranged on a surface that touches the skin. The ring includes the thermal sensation analyzing device 10 and a transmission unit that transmits analysis results via radio.

The thermal sensation analyzing system 1 can also be attached to the tip of a finger, or attached to a nail in the same manner as a false nail. That is, the thermal sensation analyzing system 1 can be attached to any part of a human body if able to measure skin temperature of a peripheral part where arteriovenous anastomoses exist. For example, the thermal sensation analyzing system 1 can be attached to a foot.

In another variation, the thermal sensation analyzing device 10 and the temperature sensor 20 can be separated. For example, the temperature sensor 20 can be in a ring form so that a person can wear it on his/her finger, while the thermal sensation analyzing device 10 can be in the form of a wristband so that a person can wear it on his/her forearm or above. It is assumed herein that the temperature sensor such as thermocouple, thermistor touches the skin. However, the use of a thermopile or thermography enables measurement of skin temperature without touching the skin.

Next is an explanation of a relationship between ripples and thermal sensation. Arteriovenous anastomoses exist in peripheral parts of the body such as hands and feet. Arteriovenous anastomoses directly link small arteries to venous plexus without through capillaries. Arteriovenous anastomoses are surrounded by muscular tissues, and when a person feels hot, the arteriovenous anastomoses dilate and increase blood flow to release heat from the body. Conversely, when the person feels cold, the arteriovenous anastomoses contract, and reduce blood flow to retain heat in the body.

When the temperature is within an appropriate range, body temperature is finely controlled by fine adjustment of blood flow through repetition of dilation and contraction of arteriovenous anastomoses. Thus, the temperature of peripheral parts changes in short duration in the vicinity of comfortable temperature along with changes in blood flow related to adjustment of body temperature.

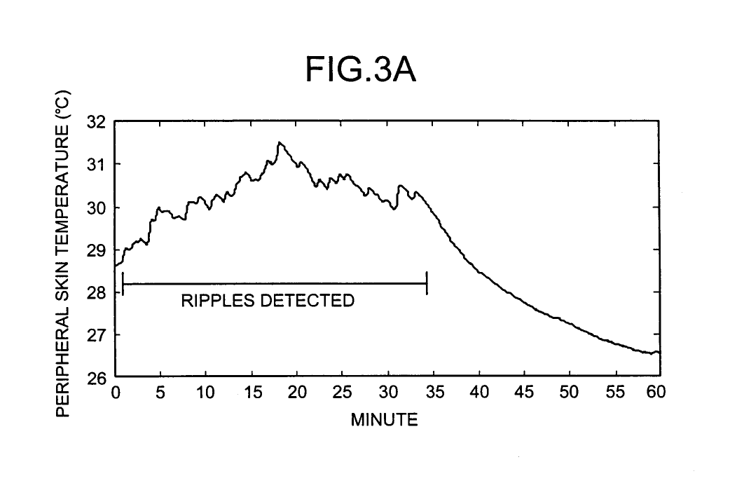

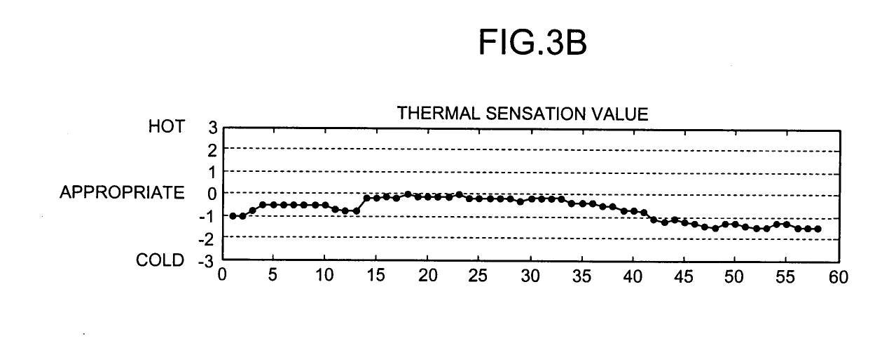

In FIG. 3A, ripples are detected during a period from 0 minute to 35 minutes. In FIG. 3B, values −1 to 1 are taken as an index of appropriate temperature environment, a value smaller than the index indicates cold temperature environment and a value larger than the index indicates hot temperature environment. In FIG. 3A, during the period when ripples are detected, a person feels comfortable. From the time when ripples are not detected, the person starts feeling cold. In other words, it is possible to judge whether the person feels comfortable through ripples.

Temperature during the experiment is maintained at 25° C. The person starts feeling cold at a point of 35 minutes, i.e., a little later after ripples are no more detected. This is because, change in feeling occurs after change in the body.

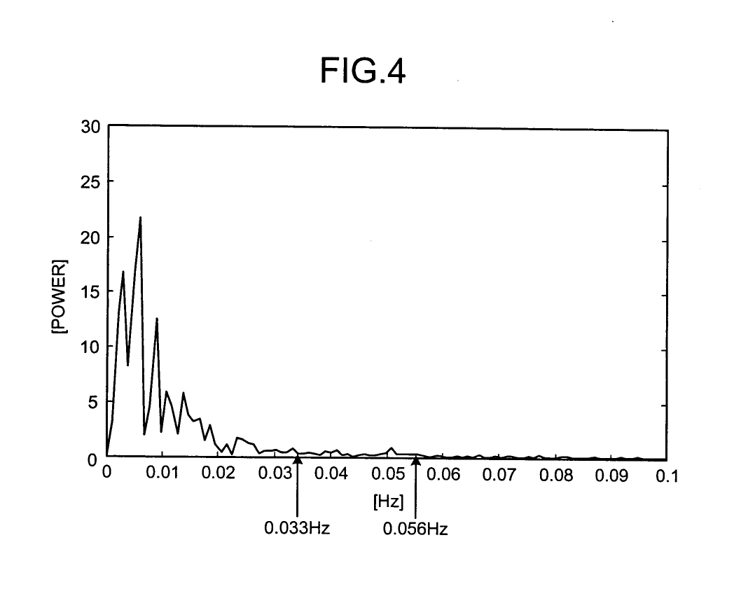

Having obtained skin temperatures as shown in FIG. 3A, the thermal sensation analyzing unit 104 obtains a power spectrum as shown in FIG. 4 by Fourier transform. From the power spectrum, for example, frequencies of 0.0056 H_{z} to 0.033 H_{z} are extracted. The peak of the frequencies of 0.0056 H_{z} to 0.033 H_{z} is compared with a predetermined threshold value. If the peak is equal to or higher than the threshold value, it is determined that there is a ripple.

A trend other than ripples is likely to be detected in a frequency range not higher than 0.0056 H_{z}. Therefore, it is preferable to extract frequencies from a range higher than 0.0056 H_{z}. The frequency of 0.0056 H_{z} corresponds to a 180 second cycle. In other words, it is desirable to detect cyclic variations of 180 seconds or less as ripples.

It is also desirable to extract frequencies from a range not higher than 0.033 H_{z}. The frequency of 0.033 H_{z} corresponds to a 30 second cycle. When temperature change was visually checked on several data samples as shown in FIG. 3B, cycles of all ripples detected were 30 seconds or more. Further, as shown in FIG. 4, according to frequency analysis, the peak is in the range not higher than 0.033 H_{z}.

As another example, in place of comparing a peak with a threshold value, an integrated value of the extracted frequencies of 0.0056 H_{z} to 0.033 H_{z} can be compared with a predetermined threshold value, and when the integrated value is equal to or larger than the threshold value, it is determined that there is a ripple.

In still another example, the ripple detection unit 102 can be a band-pass filter. In this case, frequencies from 0.0056 H_{z} to 0.033 H_{z} are used as cut-off frequencies. Amplitude of ripples derived from a filter output value is compared with a predetermined threshold value. If the amplitude is larger than the threshold value, it is determined that there is a ripple.

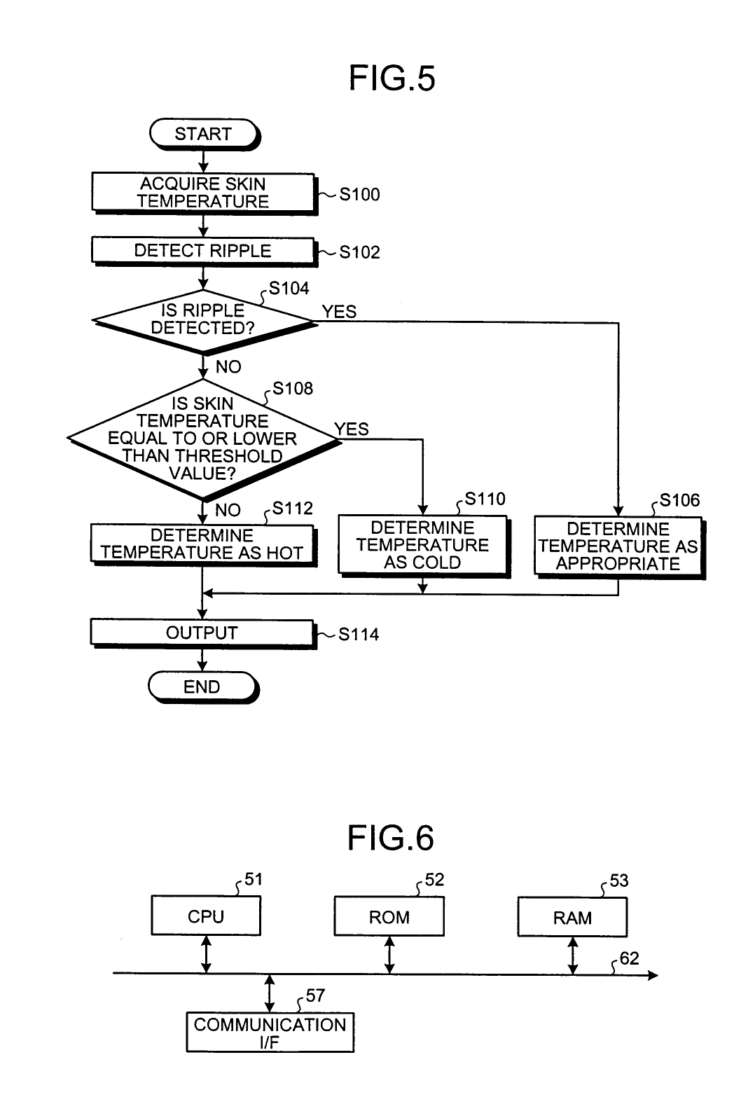

As shown in FIG. 5, in a thermal sensation analyzing process performed by the thermal sensation analyzing device 10, the skin temperature acquiring unit 100 acquires the skin temperature of a person from the temperature sensor 20 (step S100). Next, the ripple detecting unit 102 detects ripples from the skin temperature acquired by the skin temperature acquiring unit 100 (step S102). Having detected ripples (Yes at step S104), the thermal sensation analyzing unit 104 determines that the person feels comfortable, i.e., temperature is appropriate (step S106).

If ripples are not detected, temperature is not appropriate. The thermal sensation analyzing unit 104 compares the skin temperature with a predetermined threshold value. For example, the threshold value is set to 2° C. When the skin temperature is equal to or lower than the threshold value (Yes at step S108), the thermal sensation analyzing unit 104 determines that the person feels cold, i.e., temperature is cold (step S110). On the other hand, if the skin temperature exceeds the threshold value (No at S108), the thermal sensation analyzing unit 104 determines that the person feels hot, i.e., temperature is hot (step S112).

When the thermal sensation analyzing process ends, the output unit 106 outputs a thermal sensation analysis result (step S114). Thus, the thermal sensation analyzing process is completed.

As described above, according to the first embodiment, appropriateness of the temperature is determined based on the presence of ripples. Further, the skin temperature of a person is compared to a threshold value to determine whether the person feels cold or hot. Thus, it is possible to analyze thermal sensation correctly with a simple device without any hindrance in activities in daily life.

FIG. 6 is a block diagram of a hardware configuration of the thermal sensation analyzing device 10. The thermal sensation analyzing device 10 includes a central processing unit (CPU) 51, a read only memory (ROM) 52, a random access memory (RAM) 53, and a communication interface (I/F) 57, which are connected by a bus 62. The ROM 52 stores therein a computer program that implements the thermal sensation analyzing process (hereinafter, “thermal sensation analyzing program”). The CPU 51 controls each unit of the thermal sensation analyzing device 10 according to the thermal sensation analyzing program in the ROM 52. The RAM 53 stores therein various data required for controlling the thermal sensation analyzing device 10. The communication I/F 57 is connected to a network for communication.

The thermal sensation analyzing program can be stored in a computer-readable recording medium, such as a compact disk-read only memory (CD-ROM), a flexible disk (FD), and a digital versatile disk (DVD), in a form of a file that can be installed on and executed by a computer.

In this case, the thermal sensation analyzing device 10 loads the thermal sensation analyzing program from the recording medium into a main memory and executes it to implement thereon each unit explained above as software.

The thermal sensation analyzing program can also be stored in a computer that is connected to a network such as the Internet, and downloaded through the network.

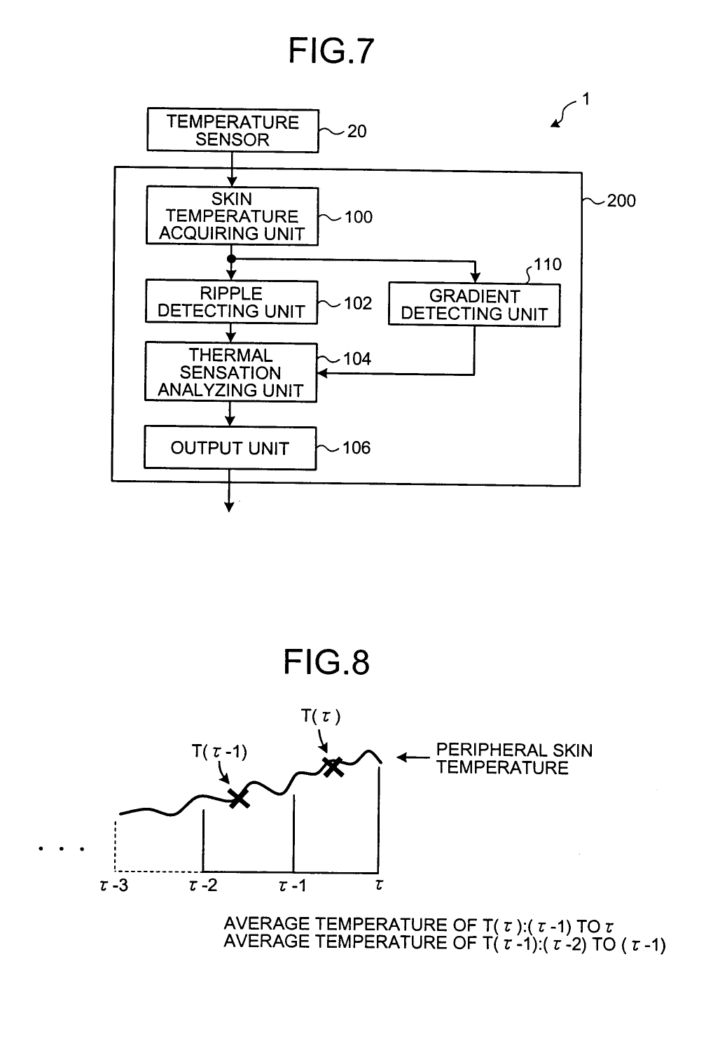

As shown in FIG. 7, a thermal sensation analyzing device 200 according to a second embodiment of the present invention is basically similar to the thermal sensation analyzing device 10 except for the presence of a gradient detecting unit 110. The gradient detecting unit 110 detects the gradient of skin temperature for a longer period than the ripple cycle used by the ripple detecting unit 102. The thermal sensation analyzing unit 104 analyzes the thermal sensation based on the gradient extracted by the gradient detecting unit 110 in addition to ripples.

As shown in FIG. 8, the gradient detecting unit 110 is, for example, a low-pass filter. The gradient detecting unit 110 calculates a difference between an average temperature T(τ) during a period from time (τ−1) to τ and an average temperature T(τ−1) during a period from time (τ−2) to (τ−1) If the difference is larger than a predetermined reference value, then it is determined that there is a gradient.

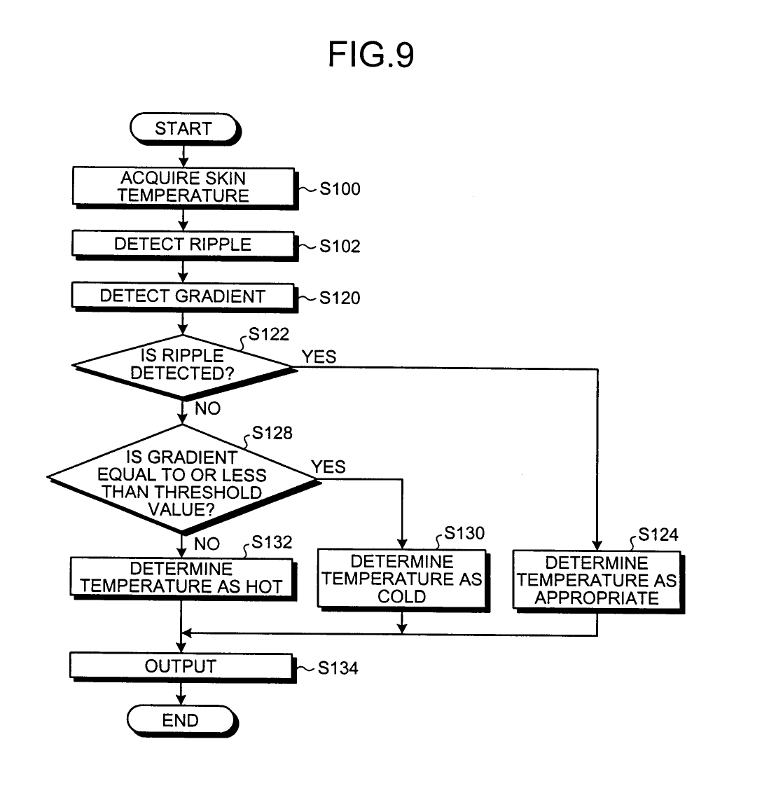

As shown in FIG. 9, in a thermal sensation analyzing process performed by the thermal sensation analyzing device 200, after acquiring the skin temperature of a person (step S100), ripples are detected (step S102), and in addition, the gradient detecting unit 110 detects a gradient (step S120). The order in which ripple detection and gradient detection are performed can be changed.

When ripples are detected (Yes at step S122), the temperature is determined to be appropriate (step S124). If ripples are not detected, in other words, if the temperature is not appropriate, whether the person feels hot or cold is determined based on the gradient. When the gradient is equal to or less than a threshold value (Yes at step S128), it is determined that the person feels cold, i.e., temperature is cold (step S130). When the gradient is higher than the threshold value (No at step S128), it is determined that the person feels hot, i.e., temperature is hot (step S132). The result of the thermal sensation analysis is output (step S134). Thus, the thermal sensation analyzing process is completed.

Otherwise, the thermal sensation analyzing system 1 according to the second embodiment is of essentially the same configuration and operates in the same manner as that of the first embodiment.

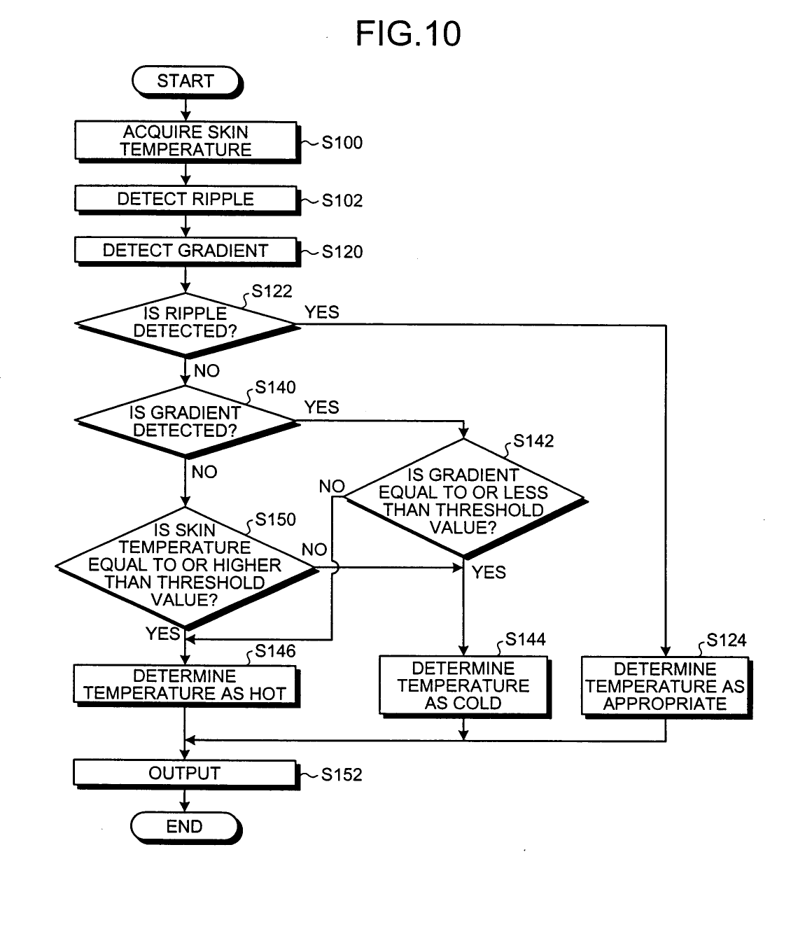

As shown in FIG. 10, a thermal sensation analyzing process performed by a thermal sensation analyzing device according to a third embodiment of the present invention is basically similar to that performed by the thermal sensation analyzing device 200, except that the thermal sensation analyzing unit 104 analyzes thermal sensation further based on the skin temperature.

The thermal sensation analyzing device according to the third embodiment checks whether there is a gradient if ripples are not detected. An arbitrary reference value is fixed, and if the gradient is more than the reference value, it is determined that there is a gradient. The reference value is smaller than a threshold value to be described later.

If there is a gradient (Yes at step S140), and the gradient is equal to or less than the threshold value (Yes at step S142), it is determined that the person feels cold, i.e., temperature is cold (step S144). On the other hand, if the gradient is higher than the threshold value (No at step S142), it is determined that the person feels hot, i.e., temperature is hot (step S146).

When there is no gradient, (No at step S140), skin temperature and a threshold value is compared. The threshold value is set to, for example, 28° C. The average value of skin temperature, acquired by the skin temperature acquiring unit 100 in a predetermined short time, is compared with the threshold value.

If the skin temperature is equal to or higher than the threshold value (Yes at step S150), it is determined that the person feels hot, i.e., temperature is hot (step S146). On the other hand, if the skin temperature is less than the threshold value (No at step S150), it is determined that the person feels cold, i.e., temperature is cold (step S144). The analysis result is output (step S152), and the thermal sensation analyzing process is completed.

Otherwise, a thermal sensation analyzing system according to the third embodiment is of essentially the same configuration and operates in the same manner as that of the second embodiment.

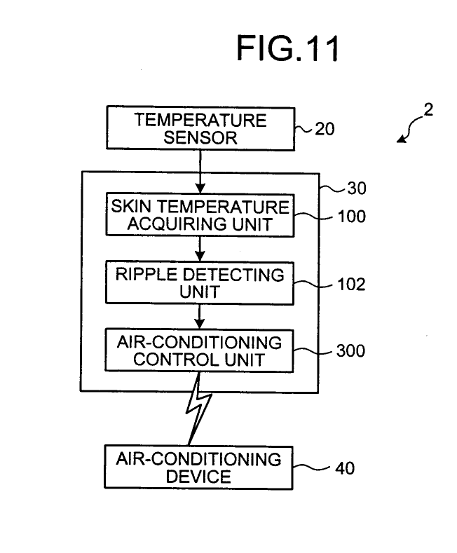

As shown in FIG. 11, an air-conditioning control system 2 according to a fourth embodiment of the present invention includes an air-conditioning control device 30, the temperature sensor 20, and an air-conditioning device 40. The air-conditioning control device 30 includes the skin temperature acquiring unit 100, the ripple detecting unit 102, and an air-conditioning control unit 300.

The air-conditioning control unit 300 controls air-conditioning by a heater or a cooler based on whether ripples are detected by the ripple detecting unit 102. Specifically, the air-conditioning device 40 is remotely controlled to turn on or off the cooler or the heater. It is also possible to remotely control the speed of a fan, a direction of the fan, in addition to turning on or off the cooler or the heater.

The process performed by the skin temperature acquiring unit 100 and the ripple detecting unit 102 is the same as previously described for the thermal sensation analyzing device 10.

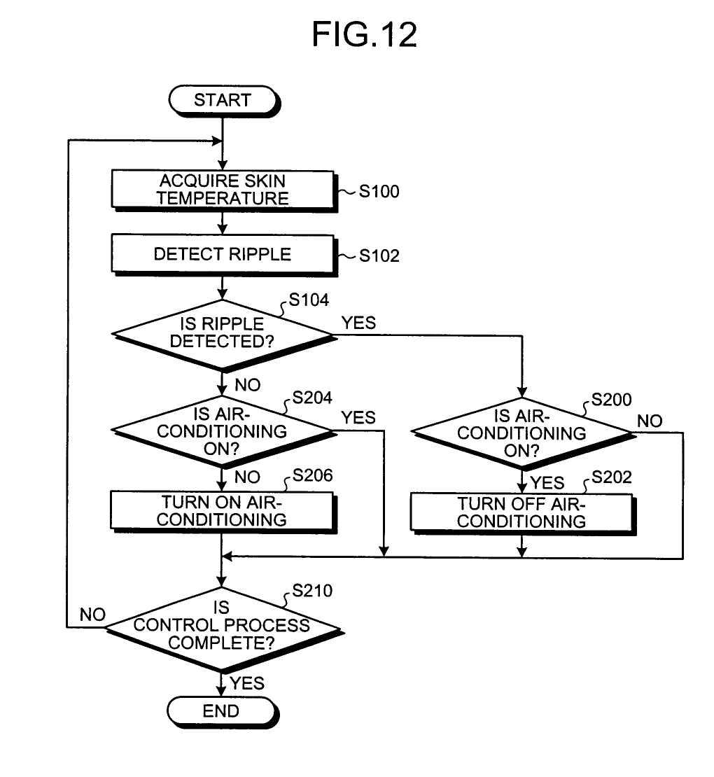

As shown in FIG. 12, in an air-conditioning process performed by the air-conditioning control device 30, after acquiring the skin temperature of a person (step S100) and detecting ripples (step S102), if ripples are detected (Yes at step S104), the air-conditioning control unit 300 checks whether air-conditioning is on or off. If the air-conditioning is on (Yes at step S200), the air-conditioning is turned off (step S202). Thus, if ripples are detected, that is, when the person feels comfortable, because room temperature is at an appropriate level, the air-conditioning is turned off.

At step S102, if ripples are not detected (No at step S104), it is checked whether the air-conditioning is on or off. If the air-conditioning is not on (No at step S204), the air-conditioning is turned on (step S206). If ripples are not detected, the person is not feeling comfortable. Therefore, the air-conditioning is turned on to adjust the room temperature to be appropriate. The process described above is repeated until an instruction to end the air-conditioning process is issued (step S210). Thus, the air-conditioning process is completed.

As described above, according to the fourth embodiment, the air-conditioning control device 30 controls turning on or off of the air-conditioning based on the presence of ripples. Therefore, it is possible to achieve effective air-conditioning. Further, minimizing operation of air conditioning contributes to saving energy.

Otherwise, the air-conditioning control system 2 is of essentially the same configuration and operates in the same manner as the thermal sensation analyzing system 1 according to the above embodiments.

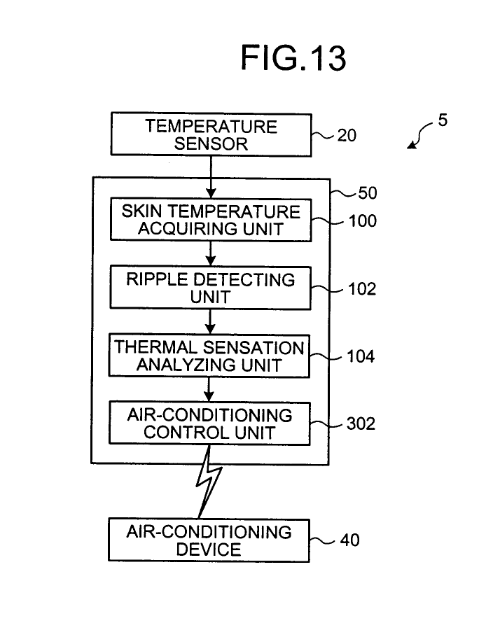

As shown in FIG. 13, an air-conditioning control system 5 according to a fifth embodiment of the present invention is basically similar to the air-conditioning control system 2 except for an air-conditioning control device 50. The air-conditioning control device 50 includes the skin temperature acquiring unit 100, the ripple detecting unit 102, the thermal sensation analyzing unit 104, and an air-conditioning control unit 302. The air-conditioning control unit 302 controls air-conditioning based on the result of analysis by the thermal sensation analyzing unit 104. Other processes are the same as previously described for the thermal sensation analyzing device according to the above embodiments.

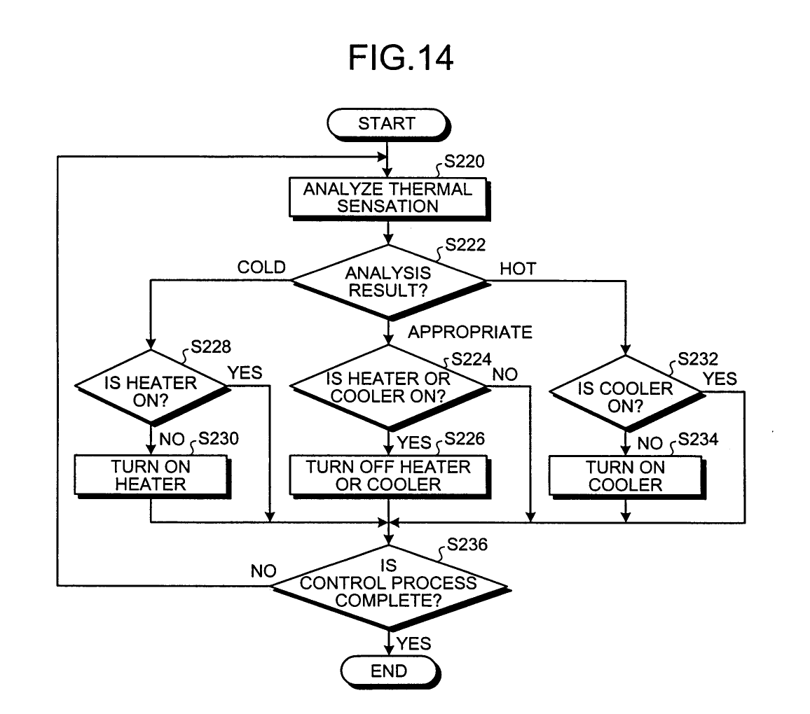

As shown in FIG. 14, in an air-conditioning process performed by the air-conditioning control device 50, thermal sensation analysis is performed through the operation of the ripple detecting unit 102 and the thermal sensation analyzing unit 104 (step S220) in the same manner as previously described in connection with FIG. 5. The air-conditioning control device 50 controls air-conditioning based on the analysis result acquired through the thermal sensation analyzing process.

Specifically, if it is determined that the person feels the temperature is appropriate (appropriate at step S222), it is checked whether the heater or the cooler is on. If the heater or the cooler is on (Yes at step S224), it is turned off (step S226).

If it is determined that the person feels cold (cold at step 222), it is checked whether the heater is on. If the heater is not on (No at step S228), the heater is turned on (step S230). If the cooler is on, the cooler is turned off and the heater is turned on.

As other examples, when it is determined that the person feels cold and the cooler is on, the cooler can just be turned off. If the heater and the cooler as well are off, the heater can be turned on.

When it is determined that the person feels hot (hot at step S222), it is checked whether the cooler is on. If the cooler is not on (No at step S232), the cooler is turned on (step S234). If the heater is on, the heater is turned off and the cooler is turned on.

As other examples, when it is determined that the person feels hot and the heater is on, the heater can just be turned off. If the heater and the cooler are off, the cooler can be turned on.

Otherwise, the air-conditioning control system 5 is of essentially the same configuration and operates in the same manner as the thermal sensation analyzing system 1 or the air-conditioning control system 2 according to the above embodiments.

Next is an explanation regarding an air-conditioning control system 6 according to a sixth embodiment of the present invention. The air-conditioning control system 6 is basically similar to the air-conditioning control system 5 except for an air-conditioning control device 60. The air-conditioning control device 60 analyzes thermal sensation based on ripples and gradient, and controls air-conditioning based on the result of the thermal sensation analysis.

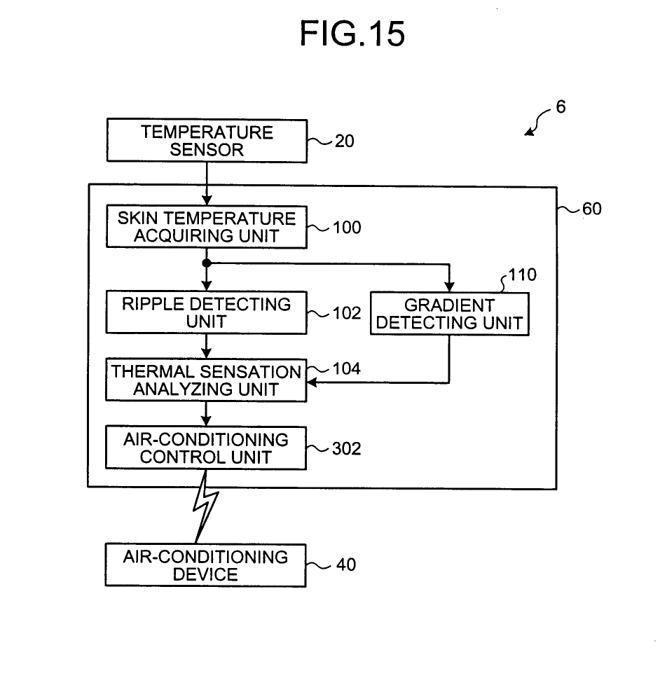

As shown in FIG. 15, the air-conditioning control device 60 includes the gradient detecting unit 110 in addition to the functional configuration of the air-conditioning control device 50. The thermal sensation analyzing unit 104 analyzes the thermal sensation based on the presence of ripples and gradient. The air-conditioning control unit 302 controls the air-conditioning based on the thermal sensation analysis result obtained by the thermal sensation analyzing unit 104.

In an air-conditioning process, the air-conditioning control device 60 first performs the thermal sensation analyzing process as explained in the second embodiment in connection with FIG. 9. Other processes are the same as previously described for the air-conditioning control device 50.

As another example, it is possible to replace the thermal sensation analyzing process of FIG. 9 with that of FIG. 10.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A thermal sensation analyzing device comprising:
a first detecting unit that detects skin temperature of a person;
a second detecting unit that detects a predetermined cyclic-fluctuation pattern in the skin temperature; and
an analyzing unit that analyzes thermal sensation of the person based on the cyclic-fluctuation pattern.

2. The device according to claim 1, wherein the analyzing unit determines temperature as appropriate when the second detecting unit detects the cyclic-fluctuation pattern.

3. The device according to claim 1, wherein the second detecting unit detects the cyclic-fluctuation pattern by analyzing frequencies with respect to the skin temperature.

4. The device according to claim 1, further comprising a comparing unit that compares the skin temperature with a threshold value, wherein
the analyzing unit analyzes the thermal sensation further based on a comparison result obtained by the comparing unit.

5. The device according to claim 1, further comprising a third detecting unit that detects a gradient of the skin temperature during a predetermined time period, wherein
the analyzing unit analyzes the thermal sensation further based on the gradient detected by the third detecting unit.

6. The device according to claim 5, wherein the analyzing unit determines that the person feels cold when the gradient is higher than a threshold value.

7. The device according to claim 6, wherein the analyzing unit determines that the person feels hot when the gradient is equal to or less than the threshold value.

8. The device according to claim 5, wherein the analyzing unit analyzes the thermal sensation further based on the skin temperature when the third detecting unit detects no gradient.

9. The device according to claim 8, wherein the analyzing unit determines that the person feels cold when the skin temperature is lower than a threshold value.

10. The device according to claim 9, wherein the analyzing unit determines that the person feels hot when the skin temperature is equal to or higher than the threshold value.

11. The device according to claim 5, wherein the second detecting unit detects cyclic fluctuation of duration shorter than the predetermined time for detecting the gradient.

12. The device according to claim 1, wherein the second detecting unit detects cyclic fluctuation of duration equal to or longer than 30 seconds.

13. The device according to claim 1, wherein the second detecting unit detects cyclic fluctuation of duration equal to or less than 180 seconds.

14. The device according to claim 1, wherein the first detecting unit detects peripheral skin temperature of the person.

15. A thermal sensation analyzing method comprising:
acquiring skin temperature of a person;
detecting a predetermined cyclic-fluctuation pattern in the skin temperature; and
analyzing thermal sensation of the person based on the cyclic-fluctuation pattern.

16. A computer program product comprising a computer usable medium having computer readable program codes for performing thermal sensation analyzing process embodied in the medium that when executed causes a computer to execute:
acquiring skin temperature of a person;
detecting a predetermined cyclic-fluctuation pattern in the skin temperature; and
analyzing thermal sensation of the person based on the cyclic-fluctuation pattern.

17. An air-conditioning control device comprising:
a first detecting unit that detects skin temperature of a person;
a second detecting unit that detects a predetermined cyclic-fluctuation pattern in the skin temperature;
an analyzing unit that analyzes thermal sensation of the person based on the cyclic-fluctuation pattern; and
an air-conditioning control unit that controls air-conditioning based on an analysis result obtained by the analyzing unit.

18. An air-conditioning control method comprising:
acquiring skin temperature of a person;
detecting a predetermined cyclic-fluctuation pattern in the skin temperature;
analyzing thermal sensation of the person based on the cyclic-fluctuation pattern; and
controlling air-conditioning based on an analysis result obtained at the analyzing.

19. A computer program product comprising a computer usable medium having computer readable program codes for performing thermal sensation analyzing process embodied in the medium that when executed causes a computer to execute:
acquiring skin temperature of a person;
detecting a predetermined cyclic-fluctuation pattern in the skin temperature;
analyzing thermal sensation of the person based on the cyclic-fluctuation pattern; and
controlling air-conditioning based on an analysis result obtained at the analyzing.
